(19) United States
(12) Patent Application Publication
(10) Pub. No.: US 2025/0325727 A1
(43) Pub. Date: Oct. 23, 2025

(54) PHOTOCURABLE ADHESIVES WITH FERULATED PECTIN

(71) Applicant: COOK BIOTECH INCORPORATED, WEST LAFAYETTE, IN (US)

(72) Inventors: SYDNEY E. HOLLINGSHEAD, LAFAYETTE, IN (US); TYLER A. NOVAK, LAFAYETTE, IN (US)

(73) Assignee: COOK BIOTECH INCORPORATED, WEST LAFAYETTE, IN (US)

(21) Appl. No.: 19/175,296

(22) Filed: Apr. 10, 2025

Related U.S. Application Data
(60) Provisional application No. 63/635,204, filed on Apr. 17, 2024.

(51) Int. Cl. A61L 24/08; A61B 17/00; A61B 17/11; A61L 24/00

(52) U.S. Cl. CPC A61L 24/08; A61B 17/1128; A61L 24/0031; A61B 2017/00526; A61B 2017/00942; A61B 2017/00951

(57) ABSTRACT

In certain embodiments, the present disclosure provides adhesive compositions and graft materials comprising covalently crosslinked hydrogels, e.g. prepared or preparable by curing the adhesive compositions. In some forms, the adhesive compositions comprise a photoactivatable catalyst such as a photoactivatable metal ligand complex, an electron acceptor, a ferulated pectin, and a liquid carrier.

### BACKGROUND

The present invention resides generally in the field of adhesive compositions and in particular aspects to medical adhesives that can be photocured.

As further background, adhesive compositions are used in a variety of medical and non-medical applications. For example, when surgical wounds on liquid-containing or gas-containing structures are closed with a suture or staple line, there is a risk of liquid or gas leakage from the closed wound site. Surgical adhesives are applied over the suture and/or staple line to reduce the risk of leakage. In other uses, surgical adhesives can be applied to bond patient tissues to one another and/or to bond implant materials to patient tissues, as well as to provide a bulking function to tissue volumes. While some work has been done in these fields, needs remain for improved and/or alternative adhesive compositions and in particular adhesive compositions that crosslink when exposed to light.

### SUMMARY

In one embodiment, the present disclosure provides a photocurable adhesive comprising a photoactivatable catalyst such as a photoactivatable metal-ligand complex, an electron acceptor, ferulated pectin, and a liquid carrier, preferably an aqueous liquid carrier. In some forms, the ferulated pectin comprises sugar beet pectin. The electron acceptor may comprise sodium persulfate, preferably at a concentration of about 10 mM. The photoactivatable metal-ligand complex may comprise ruthenium tris-bipyridyl chloride. The medical composition may further comprise one or more polymer materials other than the ferulated pectin that contain phenolic groups, for example including gelatin and/or a phenol-enriched gelatin.

In another embodiment, the present disclosure provides a method of crosslinking a medical composition. Such method comprises irradiating a composition comprising a photoactivatable catalyst such as a photoactivatable metal-ligand complex, an electron acceptor, a ferulated pectin, and a liquid medium to initiate a cross-linking reaction. In some forms, the irradiating comprises exposing the composition to visible light for a duration sufficient to initiate a cross-linking reaction, for example at least about twenty seconds. In some forms, the ferulated pectin comprises sugar beet pectin. The electron acceptor may comprise sodium persulfate, preferably at a concentration of about 10 mM. The photoactivatable metal-ligand complex may comprise ruthenium tris-bipyridyl chloride. The medical composition may further comprise gelatin, for example a phenol-enriched gelatin.

In another embodiment, the present disclosure provides a medical implant comprising a substrate suitable for implantation and a cured adhesive material carried by the substrate. The cured adhesive material comprises a crosslinked polymeric matrix including pectin and having covalent feruloyl-feruloyl crosslinks between pectin molecules. The cured adhesive material can further include a photoactivatable catalyst such as a photoactivatable metal-ligand complex and/or an electron acceptor, and can include a reaction product obtained by photocuring a photocurable liquid adhesive composition including a ferulated pectin, the photoactivatable catalyst, the electron acceptor, and a liquid medium. In some forms, the substrate comprises a membranous extracellular matrix material, for example submucosa, renal capsule membrane, dermal collagen, dura mater, pericardium, fascia lata, serosa, peritoneum or basement membrane layers, or including liver basement membrane. In certain embodiments, the membranous extracellular matrix material is in sheet form, the medical adhesive is carried on a first surface of the sheet form extracellular matrix material. In some forms, the ferulated pectin comprises sugar beet pectin. The electron acceptor may comprise sodium persulfate. The photoactivatable metal-ligand complex may comprise ruthenium tris-bipyridyl chloride.

In another embodiment, the present disclosure provides a method of preparing a photocurable liquid adhesive, the method comprising combining a ferulated pectin, a photocatalyst such as a photoactivatable metal-ligand complex, an electron acceptor, and a liquid medium. In some forms, the pectin comprises sugar beet pectin. The electron acceptor may comprise sodium persulfate, preferably at a concentration of about 10 mM. The photoactivatable metal-ligand complex may comprise ruthenium tris-bipyridyl chloride.

In yet another embodiment, the present disclosure provides a method for treating a patient that includes applying a photocurable liquid adhesive as described above and/or elsewhere herein to tissue of the patient, and photocuring the liquid adhesive. In some forms, the treatment can include joining and/or sealing tissues of the patient in a surgical procedure. Such a method can comprise applying the photocurable liquid adhesive to a tissue portion, and irradiating the liquid adhesive to initiate a cross-linking reaction to seal the tissue portion or join the tissue portion to an adjacent tissue portion. The cross-linking reaction can form covalent crosslinks within the liquid adhesive and between the liquid adhesive and endogenous proteins of the patient tissue portion(s).

Additional embodiments, as well as features and advantages of embodiments of the present disclosure, will be apparent from the descriptions herein.

### BRIEF DESCRIPTION OF THE DRAWINGS

FIG. 1 is a perspective view of one embodiment of a substrate including a medical adhesive as disclosed herein.

FIG. 2 is a graph showing the results of testing to determine the extent of polymer modification with varying concentrations of Bolton Hunter reagent as described in Example 2.

### DETAILED DESCRIPTION

For the purposes of promoting an understanding of the principles of the disclosure, reference will now be made to certain embodiments, some of which are illustrated in the drawings, and specific language will be used to describe the same. It will nevertheless be understood that no limitation of the scope of the claims is thereby intended, and alterations and modifications in the illustrated graft, and further applications of the principles of the disclosure as illustrated therein are herein contemplated as would normally occur to one skilled in the art to which the disclosure relates.

As disclosed above, aspects of the present disclosure relate to novel adhesive compositions and methods of using them. In certain aspects, the disclosure relates to photocurable liquid adhesives comprising a ferulated pectin and a photoactivatable crosslinking system such as one including a photoactivatable catalyst and an electron acceptor.

The present disclosure provides adhesive compositions that include a ferulated pectin. It has been discovered that such compositions upon photocuring advantageously form crosslinks between feruloyl groups of separate polymer molecules of the ferulated pectin (diferuloyl crosslinks). Such diferuloyl crosslinks are, more generally, crosslinks between phenolic groups of separate polymer molecules (“diphenolic crosslinks”). Where the adhesive composition also includes a polymer containing phenolic groups other than the ferulated pectin, the photocuring may also form diphenolic crosslinks between the pectin polymer molecules and molecules of the other polymer containing phenolic groups, as well as between separate polymer molecules of the other polymer containing phenolic groups. As used herein the term “phenolic group” refers to a phenyl ring having a hydroxyl group directly attached to a carbon atom of the phenyl ring. The phenyl ring can optionally have other functional groups attached thereto. For example, a feruloyl group (which has a 4-hydroxy-3-methoxyphenyl group) is a phenolic group as described herein.

In certain embodiments, the present disclosure provides methods of crosslinking ferulated pectin. Such methods comprise irradiating a photocurable liquid adhesive composition comprising a photoactivatable catalyst such as a photoactivatable metal-ligand complex and/or riboflavin, an electron acceptor, a ferulated pectin and a liquid medium, thereby initiating a cross-linking reaction. In some forms, the irradiating is conducted prior to implantation of a medical graft to form a crosslinked coating on the medical graft. The coated medical graft can then optionally be subjected to subsequent manufacturing steps, for example washing with liquid, drying, and/or sterilely packaging the graft. However, in certain embodiments the graft may be irradiated shortly before implantation, for example at the site of treatment, to create the coated graft. In such cases, a photocurable liquid adhesive as described herein may be applied to a substrate and irradiated prior to implantation. In alternative embodiments, the irradiating of the photocurable liquid adhesive is performed in situ, for example to close a wound, join tissue, and/or adhere a medical graft material to patient tissue.

The photocurable liquid adhesive will be irradiated with light at a wavelength that activates the photoactivatable catalyst and initiates the covalent crosslinking reaction. Where the photoactivatable catalyst is or includes a photoactivatable metal ligand complex as disclosed herein, preferably ruthenium tris-bipyridyl chloride, irradiation may be performed using white light (i.e. light including wavelengths between about 400 and about 700 nm). In accordance with certain embodiments, the photocurable liquid adhesive composition as described herein is cured by irradiating it for at least 5 seconds, preferably at least 10 seconds, and typically in the range of about 10 seconds to about 180 seconds, more typically in the range of about 15 seconds to about 60 seconds.

In some forms, the photocurable liquid adhesive composition will include, in addition to a ferulated pectin, one or more additional polymers containing phenolic groups. Such a polymer(s) can be a matrix protein(s). As used herein the term “matrix protein” refers to isolated and purified extracellular matrix proteins. Suitable matrix proteins for use in the compositions herein may be selected from, but not limited to the group consisting of: fibrinogen, fibrin, collagen, keratin, gelatin, fibronectin, serum albumin, elastin, beta-lactoglobulin, glycinin, glutens, gliadins, resilin and/or laminin, or admixtures thereof. Matrix proteins may be isolated from human or animal sources or can be synthetically produced for instance using recombinant techniques. In some forms, matrix proteins are isolated from ECM source tissues as described herein. In some forms, the matrix protein may be denatured to encourage the formation of diphenolic cross-links. Denaturation of a protein may be accomplished by raising or lowering the pH of a solution containing the matrix protein, decreasing or increasing the ionic strength of a solution containing the matrix protein, hydrolysis, or in other ways known to a person skilled in the art.

Turning now to a discussion of pectins, any suitable ferulated pectin may be used. As used herein “ferulated pectin” refers to pectin having feruloyl moieties. It is also advantageous to select a pectin having a polymer chain of sufficient length so as to form a solid network of polymer fibers upon crosslinking. Preferably, the ferulated pectin is derived from a natural source, for example, a plant source. Ferulated pectins have been reported, for example, from sugar beet, spinach, quinoa, glasswort and amaranth. In certain preferred embodiments, the pectin is derived from sugar beet

In accordance with some forms, the photocurable liquid adhesive can include a combination of a ferulated pectin and a matrix protein. In certain embodiments, the matrix protein has been phenol-enriched to render the matrix protein more susceptible to diphenolic cross-linking compared to its native state. By way of example primary amines such as the lysine residues in a matrix protein may be modified under mild conditions with Bolton-Hunter reagent (N-succinimidyl-3-[4hydroxyphenyl]propionate) or water-soluble Bolton-Hunter reagent (sulfosuccinimidyl-3-[4-hydroxyphenyl]propionate). In certain forms, the photocurable liquid adhesive will include a mixture of an amount of a protein (especially collagen, gelatin or a collagen peptide composition) with an amount of the corresponding phenol enriched protein

While not wishing to be bound by theory, it is believed that the crosslinking mechanism involves irradiation of the metal-ligand complex or other photoactivatable catalyst to induce an excited state, followed by transfer of an electron from the catalyst (the metal in the case of the metal-ligand complex) to the electron acceptor. The catalyst (e.g. oxidized metal of the metal-ligand complex) then extracts an electron from a feruloyl group of the ferulated pectin to produce a radical that reacts immediately with a nearby phenolic group (e.g. another feruloyl group of a separate pectin polymer molecule) to form a covalent bond. A direct cross-link (without any bridging moiety) is created quickly in this photo-initiated chemical reaction. The term “photoactivatable metal-ligand complex” as used herein means a metal-ligand complex in which the metal can enter an excited state when irradiated such that it can donate an electron to an electron acceptor in order to move to a higher oxidation state and thereafter extract an electron from a phenolic group of a polymer (e.g. a feruloyl group of a ferulated pectin) to produce a free radical without reliance upon the formation of singlet oxygen. Suitable metals include but are not limited to Ru(II), Pd(II), Cu(II), Ni(II), Mn(II) and Fe(III) in the form of a complex which can absorb light in the visible region, for example, an Ru(II) bipyridyl complex, a Pd(II) porphyrin complex, a sulfonatophenyl Mn(II) complex or a Fe(III) protoporphyrin complex, more particularly, an Ru(II) bipyridyl complex or a Pd(II) porphyrin, in particular, an Ru(II) (bpy)3 complex such as [Ru(II) (bpy)3] Cl2. Efficient cross-linking occurs in the presence of an electron acceptor, and requires only moderate intensity visible light. It has been discovered that a cross-linking reaction may occur in the absence of a photoactivatable metal-ligand complex. Such formulations may require extended curing time, for example at least two hours, and potentially up to about 24 hours. In this way, compositions of the present disclosure, with or without a metal ligand complex, may form crosslinks in the absence of light. Thus, the methods disclosed herein may be practiced without irradiating the injected composition with light, such methods require a curing time of at least two hours, and may not be fully crosslinked for about 24 hours.

As used herein the term “electron acceptor” refers to a chemical entity that accepts electron transferred to it and so refers to an easily reduced molecule (or oxidizing agent) with a redox potential sufficiently positive to facilitate the cross-linking reaction. A range of electron acceptors will be suitable. In an embodiment, the electron acceptor is a peracid, a cobalt complex, a cerium (IV) complex, or an organic acid. An exemplary reaction is shown below:

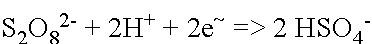

Typically, the electron acceptor is a persulfate, periodate, perbromate or perchlorate compound, vitamin B12, Co(III) (NH3)SCI2+, cerium (IV) sulphate dehydrate, ammonium cerium (IV) nitrate, oxalic acid or EDTA. Preferably, the persulfate anion is used as the electron acceptor. The standard oxidation-reduction potential for the reaction is 2.1 V, as compared to 1.8 V for hydrogen peroxide (H₂O₂). This potential is higher than the redox potential for the permanganate anion (MnO4−) at 1.7 V, but slightly lower than that of ozone at 2.2 V.

The term “phenol enriched” as applied to a polymer material herein (e.g. collagen, gelatin, or a collagen peptide composition) means that the polymer material has been chemically modified to increase the number of phenolic groups in the polymer material. Thus, “phenol enriched collagen” refers to collagen that has been chemically modified to increase the number of phenolic groups in the collagen, “phenol enriched gelatin” refers to gelatin that has been chemically modified to increase the number of phenolic groups in the gelatin, and “phenol enriched collagen peptide composition” refers to a collagen peptide composition that has been chemically modified to increase the number of phenolic groups in the collagen peptide composition. In some aspects, the phenolic groups are 4-hydroxyphenyl propionyl groups (which are much like tyrosine groups), which can be added for example using a known Bolton Hunter reagent. In some aspects, the phenol enriched polymer material (e.g. collagen, gelatin, or collagen peptide composition) will have a P/G value of at least about 7, and in certain forms in the range of about 7 to about 30, or in the range of about 15 to about 30, or in the range of about 18 to about 25, where the P/G value is the number of moles of phenol groups per mole of polymer in the polymer material. The P/G value for a polymer material can be determined using standard techniques, including for example using an absorbance assay at a wavelength of 280 nm. Moderate P/G ranges for the phenol enriched polymer, as recited above, are preferred in some aspects, as modification to higher P/G values has been found to decrease the solubility of the material in aqueous media (see e.g. Example 2 below for phenol enriched gelatin).

In preferred forms, a multi-component system is provided for preparing a photocurable adhesive as described above. A first component can include an aqueous liquid such as water or phosphate buffered saline, the ferulated pectin and if present any other polymer(s) containing phenolic groups, and the photoactivatable catalyst; and, a second component can include the electron acceptor. The second component can be in the form a dry powder or in the form of a flowable liquid, for example a flowable liquid including an aqueous medium and the electron acceptor. The first and second components can be mixed to form a flowable photocurable liquid adhesive that, when exposed to visible light, cures by the formation of covalent diphenolic crosslinks between molecules of the polymer.

Certain embodiments herein provide a kit for preparing a photocurable adhesive. The kit can include a first container defining a first chamber within a sterile barrier and containing a sterile liquid preparation in the first chamber. The sterile liquid preparation includes an aqueous liquid such as water or phosphate buffered saline, the ferulated pectin and if present any other phenolic polymer(s) dissolved in the aqueous liquid, and a photoactivatable catalyst. The kit can further include a second container defining a second chamber within a sterile barrier and containing an electron acceptor in the second chamber. The sterile liquid preparation and the electron acceptor are mixable to prepare a photocurable liquid adhesive effective to form a diphenolic crosslinked polymer hydrogel when photocured. In some forms, the kit can also include a cannulated connector for fluidly connecting the first chamber and the second chamber and/or a visible light source (e.g. a battery-powered light emitting diode visible light source) for curing the photocurable adhesive.

In some forms, the ferulated sugar beet or other pectin can have an average molecular weight (Mw) of about 10 kilodaltons to about 150 kilodaltons, more preferably about 15 #2370336 9 003433-001823 kilodaltons to about 100 kilodaltons, and typically in the range of about 15 to about 50 kilodaltons. The sterile liquid preparation can include the ferulated pectin and if present any other polymer(s) containing phenolic groups in any suitable concentration. In some forms, the total concentration of the ferulated pectin, or the ferulated pectin plus any other phenol group containing polymer(s) present in the sterile liquid preparation, will be in the range of about 1% to about 40% weight/volume, more typically about 5% to about 25% weight/volume. In beneficial forms, the sterile liquid preparation, and photocurable liquid adhesives prepared using it, can be a flowable viscous liquid, for example having a viscosity at 20° C. of greater than about 300 centipoise, or greater than about 500 centipoise, and typically in the range of about 500 to about 20000 centipoise or in the range of about 1000 to about 10000 centipoise.

The sterile liquid preparation can include the metal ligand complex in a suitable amount to catalyze the formation of covalent crosslinks in the formation of the covalently crosslinked hydrogel by photocuring. Where a Ru(II) (bpy)3 complex such as [Ru(II) (bpy)3] Cl2 is used as the metal ligand complex, preferred sterile liquid preparations will include it at a concentration in the range of about 0.2 to about 2 mM, more desirably about 0.4 to about 1 mM. Where the electron acceptor to be mixed with the sterile liquid preparation is in dry powder form, the prepared photocurable liquid adhesive will have these same concentrations of the metal ligand complex. Where the electron acceptor is provided in a solution to be combined with the sterile liquid preparation, the concentration of the metal ligand complex in the prepared photocurable liquid adhesive will be reduced relative to that in the sterile liquid preparation. In some such forms, the volume of the sterile liquid preparation, the volume of the solution of electron acceptor, and the concentration of the metal ligand complex in the sterile liquid preparation, can be selected to provide a concentration of the metal ligand complex in the prepared photocurable liquid adhesive that is within the above-referenced concentration range values given for the sterile liquid preparation.

The sterile liquid preparation can have been terminally sterilized within the first chamber to render the liquid preparation sterile (e.g. using sterilizing radiation applied to a package containing the first container), but in some preferred forms the liquid preparation is sterilely prepared, for example including passage of the liquid preparation through a sterile filter, and then filled into the first chamber in a sterile filling operation. Such sterilely-filled liquid preparations in the first chamber can therefore be free from exposure to sterilizing radiation, and thus can be free from any degradation of the ferulated pectin and/or any other polymer(s) containing phenolic groups caused by the sterilizing radiation. In some forms, the liquid preparation can be in a heated condition to reduce its viscosity during passage through the sterile filter. Also, in some forms, the first container having the first chamber containing the sterilely-filled liquid preparation can be sealed within a sterile barrier package under sterile conditions. Further, such sterile barrier package is preferably impermeable to visible light, as can be provided for example by a foil pouch package.

In some forms, the present disclosure provides a medical implant graft comprising a substrate material and a photocurable liquid adhesive as disclosed herein carried by the substrate material, or comprising a substrate material and a cured hydrogel material prepared or preparable by photocuring a photocurable liquid adhesive as disclosed herein. For instance, the photocurable liquid adhesive or the cured hydrogel material can be coated on and/or incorporated within the substrate material. In certain embodiments, such substrate materials can be in the form of a medical wrap or overlay. In certain embodiments, the substrate material comprises a remodelable material. Particular advantage can be provided by including a remodelable collagenous material in or as the substrate material. Such remodelable collagenous materials can be provided, for example, by collagenous materials isolated from a suitable tissue source from a warm-blooded vertebrate, and especially a mammal. Reconstituted or naturally derived collagenous materials can be used in the present invention. Such materials that are at least bioresorbable will provide advantage in the present invention, with materials that are bioremodelable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth. In some forms, the substrate material comprises a surgical mesh. The substrate may comprise a synthetic material. Suitable synthetic materials include non-bioresorbable or bioresorbable synthetic polymer materials such as polytetrafluroethylene (PTFE, e.g. GORE-TEX material), nylon, polypropylene, polyurethane, silicone, DACRON polymer, polyglycolic acid (PGA), polylactic acid (PLA), polycaprolactone, or others. In some forms, the substrate material may include a collagenous extracellular matrix material and a synthetic material. For example, a synthetic polymer material may be used to stitch layers of collagenous extracellular matrix materials together, or to reinforce one or more layers of collagenous extracellular matrix material. In certain embodiments, a synthetic mesh may be present alongside, or between layers of collagenous extracellular matrix materials.

Suitable bioremodelable materials can be provided by collagenous extracellular matrix materials (ECMs) possessing biotropic properties, including in certain forms angiogenic collagenous extracellular matrix materials. For example, ECMs include materials such as submucosa, renal capsule membrane, dermal collagen, dura mater, pericardium, fascia lata, serosa, peritoneum or basement membrane layers, including liver basement membrane. Suitable submucosa-containing materials for these purposes include, for instance, membranous materials that include intestinal submucosa, including small intestinal submucosa, stomach submucosa, urinary bladder submucosa, and uterine submucosa. These identified submucosa or other layers can occur in the ECM material alone, or in combination with other materials such as those derived from one or more adjacent layers in the source tissue.

The submucosa-containing ECM can be derived from any suitable organ or other biological structure, including for example submucosa derived from the alimentary, respiratory, intestinal, urinary or genital tracts of warm-blooded vertebrates. Submucosa-containing materials useful in the present invention can be obtained by harvesting such tissue sources and delaminating the submucosa (alone or combined with other materials) from smooth muscle layers, mucosal layers, and/or other layers occurring in the tissue source. For additional information as to submucosal materials useful in the present invention, and its isolation and treatment, reference can be made, for example, to U.S. Pat. Nos. 4,902,508, 5,554,389, 5,993,844, 6,206,931, and 6,099,567.

As prepared, the submucosal material and any other ECM used may optionally retain growth factors or other bioactive components native to the source tissue. For example, the submucosal or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a native bioactive component that induces, directly or indirectly, a cellular response such as a change in cell morphology, proliferation, growth, protein or gene expression.

Submucosal or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

Further, in addition or as an alternative to the inclusion of native bioactive components, non-native bioactive components such as those synthetically produced by recombinant technology or other methods, may be incorporated into the submucosal or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient. Alternatively, or additionally, a non-native bioactive component can be included in the coating material of the medical product. When included in the coating, the non-native bioactive component can be added at any point during preparation of the medical product, including being mixed with one or all of the coating components prior to application of the coating to a surface of a layer of a medical material or, alternatively, after the coating is formed, applied, or cross-linked.

A non-native bioactive component can be applied to a submucosal or other ECM tissue by any suitable means. Suitable means include, for example, spraying, impregnating, dipping, etc. The non-native bioactive component can be applied to the ECM tissue either before or after the coating is applied to the material, or both. Similarly, if other chemical or biological components are included in the ECM tissue, the non-native bioactive component can be applied either before, in conjunction with, or after these other components.

Submucosal or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosal or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosal tissue used in the present invention.

In some embodiments herein, the medical implant graft can be a multilaminate medical graft that carries a photocurable liquid adhesive as described herein or a cured hydrogel material prepared or preparable by photocuring a photocurable liquid adhesive as described herein. For example, a plurality of (i.e. two or more) layers of a biocompatible material, for example submucosa-containing or other ECM material, can be bonded together to form a multilaminate structure. Illustratively, two, three, four, five, six, seven, or eight or more layers of a biocompatible material can be bonded together to provide a multilaminate bolster material. The layers of biocompatible material can be bonded together in any suitable fashion, including dehydrothermal bonding under heated, non-heated or lyophilization conditions, stitching, using a photocurable adhesive as described herein, glues or other bonding agents, crosslinking with chemical agents or radiation (including UV radiation), or any combination of these with each other or other suitable methods.

In accordance with some forms, the photocurable liquid adhesive compositions described herein may include one or more additives that alter the performance of the composition. Suitable additives for use herein may be included to improve the lubricity or reduce any inflammatory response of patient tissue to the cross-linked hydrogel material formed by curing the liquid adhesive. In certain embodiments, the additive for increasing the lubricity of the crosslinked hydrogel material include but are not limited to: hyaluronic acid, sodium hyaluronate, and/or chondroitin sulfate. In some forms one or more hydrophilic additives may be included. Suitable hydrophilic additives include sugars, for example fructose. Hydrophilic additives may cause the liquid adhesive to form a more robust layer upon crosslinking. In some forms, one or more additives may be included, which contribute additional resistance to adhesion of the crosslinked adhesive to surrounding patient tissues, for example one or more zwitterionic polymers.

The present disclosure provides methods of making an implantable medical graft. In some forms, the present disclosure provides methods including the step of applying a liquid adhesive composition as described herein to a substrate as described herein. Such applying can be achieved in any suitable fashion, for example spraying, brushing, soaking, rolling, injecting, or any other suitable technique. After the liquid adhesive composition is applied to the substrate, the resulting construct may then be irradiated to form a medical implant material comprising a crosslinked ferulated pectin-containing hydrogel. Thus, in accordance with some forms, methods of the present disclosure may include the step of irradiating a substrate as described herein.

The implantable medical grafts described herein have broad application. In some aspects, inventive products will find use as precursor materials for the later formation of a variety of other medical products, or components thereof. Medical grafts and materials that are already commercially available can be modified in accordance with the present invention as well. In certain embodiments, inventive products are useful in procedures to replace, augment, support, repair, and/or otherwise suitably treat diseased or otherwise damaged or defective patient tissue. Some of the illustrative implantable medical grafts described herein will be useful, for example, in treating diseased or damaged nerve tissue and grafts as disclosed herein can be developed and used in many other medical contexts. In this regard, when used as a medical graft, the devices disclosed herein can be utilized in any procedure where the application of the graft to a bodily structure provides benefit to the patient. Illustratively, graft materials of the invention can be processed into various shapes and configurations, for example, into a variety of differently shaped urethral slings, surgical bolster or reinforcement materials (e.g., for use in tissue resection and similar procedures), wound products and other grafts and graft-like materials.

In certain embodiments, the liquid adhesive is present in a uniform layer covering one or more surfaces of the underlying substrate. In some forms, the substrate is generally sheet-form having a first surface and a second surface. In certain embodiments, the medical adhesive is present on the first surface while the second surface is free of medical adhesive. In some forms, the medical adhesive is present on both the first a second surfaces. In accordance with certain embodiments, the medical adhesive is soaked into the substrate, such that the medical adhesive permeates the matrix structure of the substrate prior to crosslinking. The medical adhesive can be present in a variety of forms, for example in certain embodiments the medical adhesive is patterned on the surface of the substrate. The medical adhesive may be present in any suitable pattern, for example lines, cross-hatching, dots, or dots. Thus, in some forms a surface may have one or more coated portions and one or more uncoated portions. Such patterns may be advantageous, for example, to allow portions of the substrate to contact patient tissue, or to promote tissue sealing to only a portion of the substrate.

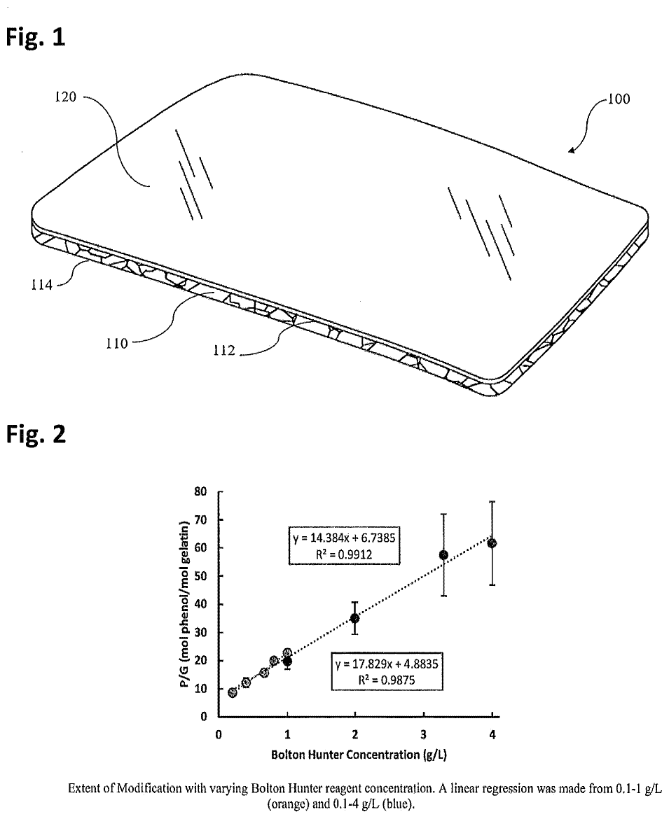

With reference now to the embodiment depicted in FIG. 1, shown is one embodiment of an implantable medical graft 100. In the illustrated embodiment, the medical graft comprises a substrate 110 and an adhesive material 120. In the illustrated embodiment, the adhesive material is coated in a substantially uniform layer disposed on a first face 112 of the substrate. A second face 114 of the substrate is free of the adhesive material. As shown, the adhesive material is layered on at least one surface of the substrate, however as described herein it is within the scope of the disclosure to provide a substrate material and an adhesive in any suitable form, e.g. partially coated, fully coated, saturated, etc. In the illustrated embodiment, the substrate and the coating are present in substantially sheet form.

In some forms, the liquid adhesive composition as described herein or a cross-linked hydrogel prepared or preparable by photocuring the liquid adhesive may be present in a uniform layer over substantially all of a coated a face of a substrate material. In other embodiments, the liquid adhesive or cross-linked hydrogel is patterned unto the substrate face such that the face of the substrate material has coated portions and uncoated portions. The liquid adhesive or cross-linked hydrogel may be present in any suitable pattern, for example, linear segments extending from one end of the graft to the other. In some forms, the liquid adhesive or cross-linked hydrogel is present in shaped sections, such as one or more circular or polygonal shaped coated portions on the surface of the substrate.

It is also within the scope of the present disclosure to provide a medical graft material comprising the liquid adhesive or the cross-linked hydrogel on both faces of a sheet-form substrate material. For example, a substrate material may be provided having a first face opposing a second face, and a first liquid adhesive or cross-linked hydrogel layer is present on the first face and a second liquid adhesive or cross-linked hydrogel layer is present on the second face. As disclosed above the liquid adhesive or cross-linked hydrogel layers may coat the entire face or only a coated portion leaving uncoated portions. The two faces may be coated in the same fashion, e.g. each having a uniform or patterned coating. Alternatively, the two faces may be coated differently, for example, a first surface may have a uniform coating while the second face has a patterned coating.

To promote a further understanding of embodiments disclosed herein and their features and advantages, the following specific Examples are provided. It will be understood that these examples are illustrative and not limiting in nature.

### Example 1

An adhesive composition was formed by mixing Sugar beet pectin in aqueous solution and combined with an oxidizer (sodium persulfate) and a photo-catalyst (ruthenium). The adhesive composition was formed comprising 7.9 w % sugar beet pectin, in 0.1M sodium citrate buffer with 0.9 mM Tris(bipyridine)ruthenium(II) chloride, and 10 mM sodium persulfate. The resulting solution was exposed to a xenon operating lamp for 30 seconds at 100% power, and solidified into a solid. A control solution prepared at the same time in the same manner was covered with foil (protected from light exposure) and did not solidify. These results indicate that the solidification was due to light-triggered crosslinking.

### Example 2

Phenol Enriched Gelatin Prepared with Bolton Hunter Reagent

Nippi MediGelatin (derived from porcine skin; Mw approximately 100 kilodaltons) was dissolved in high purity water with 6.18 g/L boric acid, 9.54 g/L sodium borate, and 4.38 g/L sodium chloride at 30° C. and 60° C. at a concentration of 10 g/L in a 1 L reaction volume (n=1). 200 mL aliquots of this solution were extracted into 500 mL Erlenmeyer flasks and combined with 3.5 mL of a Bolton Hunter reagent/DMSO solution (Bolton Hunter reagent=N-succinimidyl-3-[4-hydroxyphenyl]propionate). The Bolton Hunter/DMSO solution was prepared at a concentration such that the final concentration of Bolton Hunter reagent in the gelatin solution ranged from 0.2 to 5 g/L. The mixture was reacted at 40° C. in a shaken incubator for two hours. The solution was dialyzed, dried, and analyzed for phenol content using absorbance at 280 nm. Unless noted otherwise, groups had a replicate size of n=3. Each group was evaluated for normality and compared across groups for equal variance. Groups were then compared for statistical difference using a one-way ANOVA (α=0.05) and Tukey post-hoc tests.

The results are summarized in FIG. 2. P/G values (mole phenol/mole gelatin) for the modified materials ranged from just under 10 to about 60 (the unmodified gelatin had a P/G value of about 3). At all concentrations at and below 1 g/L Bolton Hunter, the resulting phenol enriched gelatin was soluble in PBS. The relationship between Bolton Hunter concentration and extent of phenol enrichment was linear, with a 10% change in Bolton Hunter concentration causing a ˜6% change in the molar ratio of phenol to gelatin. At Bolton Hunter concentrations at 2 g/L and higher, the relationship became less linear and the measurements had higher standard deviation. In addition, the modified gelatin became less soluble in PBS, with protein modified at 5 g/L becoming completely insoluble. These results indicate that a severe excess of Bolton Hunter reagent and a resulting very high P/G value for the phenol enriched protein can decrease the solubility of the modified protein in an aqueous medium.

A phenol-enriched gelatin as prepared in this Example can be used in combination with a ferulated pectin in a liquid adhesive as disclosed herein.

### Example 3

### Phenol Enriched Gelatin Prepared by Carbodiimide Reaction

Gelatin was modified to include additional phenol groups using EDC (1-ethyl-3-(3-dimethylaminopropyl) carbodiimide hydrochloride), NHS (N-hydroxysuccinimide), and HPPA (3-(4-Hydroxyphenyl) propionic acid). A precipitate was first prepared using a 5:2:1 ratio of NHS:HDC:HPPA at concentrations of 325 mM NHS, 130 mM EDC and 65 mM HPPA. First, HPPA was solubilized in a 0.1M MES, 0.9% Sodium Chloride, pH 4.7 buffer on a stir plate at 200 rpm. Once the HPPA was dissolved, the EDC and NHS were added to the solution. After 15-20 minutes a precipitate began to form. The solution was allowed to react for 2 to 4 hours and then vacuum filtered. Following double filtration of the solution, the precipitate captured on the filter paper was allowed to dry in a fume hood for at least 24 hours. Once the precipitate was dry, it was utilized as solubilized in DMSO in 4× mass in place of the Bolton-Hunter Reagent in a reaction conducted generally as in Example 2 to add phenolic groups to the gelatin.

A phenol-enriched gelatin as prepared in this Example can be used in combination with a ferulated pectin in a liquid adhesive as disclosed herein.

The use of the terms “a” and “an” and “the” and similar referents in the context of describing the invention especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EMBODIMENTS

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.

1. A photocurable liquid adhesive composition comprising:
a photoactivatable catalyst;
an electron acceptor;
a ferulated pectin; and
a liquid carrier.

2. The liquid adhesive composition of embodiment 1, wherein said ferulated pectin comprises sugar beet pectin.

3. The liquid adhesive composition of any one of the preceding embodiments, wherein said electron acceptor comprises sodium persulfate.

4. The liquid adhesive composition of embodiment 3, wherein the photoactivatable catalyst comprises a photoactivatable metal ligand complex.

5. The liquid adhesive composition of any one of the preceding embodiments, further comprising one or more phenolic group containing polymer other than the ferulated pectin.

6. The liquid adhesive composition of any one of the preceding embodiments, wherein the ferulated pectin is present at a concentration in the range of about 5 to about 25% by weight.

7. The medical composition of any one of the preceding embodiments, wherein said photoactivatable catalyst comprises ruthenium tris-bipyridyl chloride.

8. A method of forming a covalently crosslinked hydrogel, the method comprising:
irradiating a composition comprising a photoactivatable catalyst, an electron acceptor, a ferulated pectin and a liquid carrier to initiate a cross-linking reaction.

9. The method of embodiment 8, wherein said irradiating comprises exposing the composition to visible light.

10. The method of embodiment 9, wherein said exposing is for a duration of at least 10 seconds.

11. The method of embodiment 10, wherein the duration is in the range of 10 to 120 seconds.

12. The method of any one of embodiments 8 through 11 wherein the ferulated pectin comprises sugar beet pectin.

13. The method of any one of embodiments 8 through 12, wherein the electron acceptor comprises sodium persulfate.

14. The method of embodiment 13, wherein the photoactivable catalyst comprises a photoactivatable metal ligand complex.

15. The method of any one of embodiments 8 through 14, wherein the composition further comprises one or more polymers containing phenolic groups other than the ferulated pectin.

16. The method of any one of embodiments 8 through 15, wherein the ferulated pectin is present in the composition at a concentration in the range of about 5 to about 25% by weight.

17. The method of any one of embodiments 8 through 16, wherein the photoactivatable catalyst comprises ruthenium tris-bipyridyl chloride.

18. The method of any one of embodiments 8 through 17, wherein the composition is in contact with a biocompatible substrate during said irradiating.

19. The method of any one of embodiments 8 through 16, wherein the composition is in contact with tissue of a patient during said irradiating.

20. A medical implant comprising:
a biocompatible substrate;
a covalently crosslinked, ferulated pectin-containing hydrogel carried by said substrate, said covalently crosslinked, ferulated pectin-containing hydrogel including covalent diferuloyl crosslinks between polymer molecules of the ferulated pectin.

21. The medical implant of embodiment 20, wherein the covalently crosslinked, ferulated pectin-containing hydrogel is prepared or preparable by a process including photocuring a liquid adhesive comprising a photoactivatable catalyst, an electron acceptor, a ferulated pectin, and a liquid carrier.

22. The medical implant of embodiment 21, wherein said substrate comprises an extracellular matrix material.

23. The medical implant of embodiment 22, wherein said extracellular matrix material comprises submucosa, renal capsule membrane, dermal collagen, dura mater, pericardium, fascia lata, serosa, peritoneum or basement membrane layers, or including liver basement membrane.

24. The medical implant of any one of embodiments 20 through 23, wherein the covalently crosslinked, ferulated pectin-containing hydrogel includes an amount of a photoactivatable catalyst.

25. The medical implant of any one of embodiments 20 through 24, wherein the ferulated pectin comprises sugar beet pectin.

26. The medical implant of any one of embodiments 20 through 25, wherein the covalently crosslinked, ferulated pectin-containing hydrogel incudes an amount of an electron acceptor.

27. The medical implant of embodiment 26, wherein said electron acceptor comprises sodium persulfate.

28. The medical implant of any one of claims 20 through 27, wherein the covalently crosslinked, ferulated pectin-containing hydrogel also includes one or more polymers containing phenolic groups other than the ferulated pectin.

29. The medical implant of embodiment 28, wherein the covalently crosslinked, ferulated pectin-containing hydrogel is in dehydrated condition.

30. The medical implant of any one of embodiments 20 through 29, wherein the covalently crosslinked, ferulated pectin-containing hydrogel includes an amount of ruthenium tris-bipyridyl chloride.

31. A method of preparing a photocurable liquid adhesive, the method comprising:
combining a ferulated pectin, a photoactivatable catalyst, an electron acceptor, and a liquid carrier.

32. The method of embodiment 31, wherein the ferulated pectin comprises sugar beet pectin.

33. The method of any one of embodiments 31 or 32, wherein the electron acceptor comprises sodium persulfate.

34. The method of embodiment 33, wherein the photoactivatable catalyst comprises a photoactivatable metal ligand complex.

35. The method of embodiment 34, wherein the photoactivatable metal-ligand complex comprises ruthenium tris-bipyridyl chloride.

36. The method of any one of embodiment 31 through 35, further comprising:
applying the photocurable adhesive to a biocompatible substrate.

37. The method of embodiment 36, wherein the substrate comprises a membranous extracellular matrix material.

Any or all of the components described herein can be provided in a sterile pack for providing necessary parts, or a variety of parts, to a surgeon. For example, one or more medical grafts may be provided in a single sterile package or kit, including an adhesive composition. The adhesive composition may be pre-applied on the medical graft material or may be applied by the surgeon prior to implantation. A surgeon can choose the sizes or types of medical graft he or she wishes to use during surgery. For example, a surgeon may modify the size and/or shape of the medical graft prior to implantation. Alternatively, sterile kits containing predetermined sizes or types of components may be provided. Packages or kits of the components described herein can include additional devices or tools, which may be useful in the particular medical procedure being performed.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A photocurable liquid adhesive composition comprising:
a photoactivatable catalyst;
an electron acceptor;
a ferulated pectin; and
a liquid carrier.

2. The liquid adhesive composition of claim 1, wherein said ferulated pectin comprises sugar beet pectin.

3-4. (canceled)

5. The liquid adhesive composition of claim 1, further comprising one or more phenolic group containing polymer other than the ferulated pectin.

6. The liquid adhesive composition of claim 1, wherein the ferulated pectin is present at a concentration in the range of about 5 to about 25% by weight.

7. (canceled)

8. A method of forming a covalently crosslinked hydrogel, the method comprising:
irradiating a composition comprising a photoactivatable catalyst, an electron acceptor, a ferulated pectin and a liquid carrier to initiate a cross-linking reaction.

9. The method of claim 8, wherein said irradiating comprises exposing the composition to visible light.

10. The method of claim 9, wherein the exposing is for a duration of at least 10 seconds.

11. The method of claim 10, wherein the duration is in the range of 10 to 120 seconds.

12. The method of claim 8 wherein the ferulated pectin comprises sugar beet pectin.

13-14. (canceled)

15. The method of claim 8, wherein the composition further comprises one or more polymers containing phenolic groups other than the ferulated pectin.

16. The method of claim 8, wherein the ferulated pectin is present in the composition at a concentration in the range of about 5 to about 25% by weight.

17. (canceled)

18. The method of claim 8, wherein the composition is in contact with a biocompatible substrate during said irradiating.

19. The method of claim 8, wherein the composition is in contact with tissue of a patient during said irradiating.

20. A medical implant comprising:
a biocompatible substrate;
a covalently crosslinked, ferulated pectin-containing hydrogel carried by said substrate, said covalently crosslinked, ferulated pectin-containing hydrogel including covalent diferuloyl crosslinks between polymer molecules of the ferulated pectin.

21. The medical implant of claim 20, wherein the covalently crosslinked, ferulated pectin-containing hydrogel is prepared or preparable by a process including photocuring a liquid adhesive comprising a photoactivatable catalyst, an electron acceptor, a ferulated pectin, and a liquid carrier.

22. The medical implant of claim 21, wherein said substrate comprises an extracellular matrix material.

23. (canceled)

24. The medical implant of claim 20, wherein the covalently crosslinked, ferulated pectin-containing hydrogel includes an amount of a photoactivatable catalyst.

25. The medical implant of claim 20, wherein the ferulated pectin comprises sugar beet pectin.

26. The medical implant of claim 20, wherein the covalently crosslinked, ferulated pectin-containing hydrogel incudes an amount of an electron acceptor.

27. (canceled)

28. The medical implant of claim 20, wherein the covalently crosslinked, ferulated pectin-containing hydrogel also includes one or more polymers containing phenolic groups other than the ferulated pectin.

29. The medical implant of claim 28, wherein the covalently crosslinked, ferulated pectin-containing hydrogel is in dehydrated condition.

30. (canceled)

31. A method of preparing a photocurable liquid adhesive, the method comprising:
combining a ferulated pectin, a photoactivatable catalyst, an electron acceptor, and a liquid carrier.

32. The method of claim 31, wherein the ferulated pectin comprises sugar beet pectin.

33. (canceled)

34. The method of claim 31, wherein the photoactivatable catalyst comprises a photoactivatable metal ligand complex.

35. (canceled)

36. The method of claim 31, further comprising:
applying the photocurable adhesive to a biocompatible substrate.

37. The method of claim 36, wherein the substrate comprises a membranous extracellular matrix material.
